(19) United States
(12) Patent Application Publication
(10) Pub. No.: US 2005/0177213 A1
(43) Pub. Date: Aug. 11, 2005

(54) DISPOSABLE CRYOTHERAPY DEVICE FOR THE TREATMENT OF HEMORRHOIDS WITH FROZEN HEALING MEDIA

(76) Inventor: Jerzy Pohler, Toronto (CA)

Correspondence Address: MARKS & CLERK, 1075 NORTH SERVICE ROAD WEST, SUITE 203, OAKVILLE, ON, L6M 2G2, CA

(21) Appl. No.: 10/775,362

(22) Filed: Feb. 10, 2004

(51) Int. Cl. A61F 7/00; A61F 7/12

(52) U.S. Cl. 607/105; 607/108; 607/113

(57) ABSTRACT

The present invention provides a disposable cryotherapy device for the treatment of hemorrhoids. The cryotherapy device includes a hollow rectal insert with a wall that defines a plurality of slots. The device further includes a sleeve with an open end such that the rectal insert can slide into the sleeve. The sleeve covers the slots defined by the wall of the rectal insert. A frozen healing medium is located within the hollow rectal insert and is preferably formulated to provide about 5 minutes of cooling prior to melting during treatment of hemorrhoids or rectal tissue. The rectal insert is substantially cylindrical in shape and has a rounded end. The disposable cryotherapy device is preferably individually packaged. In another embodiment, the disposable cryotherapy device is an external applicator.

### FIELD OF THE INVENTION

This invention relates to devices for the treatment of hemorrhoids and rectal tissue.

### BACKGROUND OF THE INVENTION

Many approaches have been made to the treatment of hemorrhoids and rectal tissue following surgical procedures over the years with varying degrees of success in each case. One approach has been the use of a cold temperature rectal insert for treating the hemorrhoids or rectal tissue and to promote healing process after surgery. Another approach has been to provide medicants to the hemorrhoids or rectal tissue.

U.S. Pat. No. 3,393,842 to Arthur M. Harris teaches a frozen disposable rectal insert. The rectal insert of this reference does not deliver a pharmacological healing treatment to rectal tissue being treated.

U.S. Pat. No. 4,563,182 to Vladimir A. Stoy, et al. teaches a rectal insert that is made of a frozen gel that may dissolve over time. Because the insert is made of a gel that may dissolve, the shape of the insert may change and thus become difficult to retain within the rectum. Alternatively, the shape of the insert may change such that it is difficult to remove the insert from the rectum once the treatment is finished. This reference further teaches that the gel is “entirely without physiological effect” and thus is limited to thermal treatment.

U.S. Pat. No. 5,178,627 to Harriet Hudock teaches a rectal insert that includes pores containing medicants that may be released during treatment. This reference teaches a device that does not provide a cryotherapy effect and does not teach a delay prior to releasing the medicants.

It is desired in the art to have a device that provides a cryotherapy effect prior to delivering a healing medium.

Therefore, a device that provides first a controlled cryotherapy effect and then a healing medium to hemorrhoids or rectal tissue after surgery would be useful and provide therapeutic advantages otherwise not available.

Further, a disposable device that provides a frozen healing medium and that is both convenient to use and comfortable is desired in the art.

### SUMMARY OF THE INVENTION

The invention comprises a disposable cryotherapy device for the treatment of hemorrhoids. The cryotherapy device includes a hollow rectal insert with a wall that defines a plurality of slots. The device further includes a sleeve with an open end such that the rectal insert can slide into the sleeve. The sleeve covers the slots defined by the wall of the rectal insert. A frozen healing medium is located within the hollow rectal insert and is preferably formulated to provide about 5 minutes of cooling prior to melting during treatment of hemorrhoids or rectal tissue. The rectal insert is substantially cylindrical in shape and has a rounded end. The disposable cryotherapy device is preferably individually packaged.

In another embodiment, the disposable cryotherapy device is an external applicator.

An advantage of the present invention is that the cryotherapy device provides a frozen healing medium that first provides a cryotherapy effect and then delivers a healing medium to hemorrhoids or rectal tissue after surgery.

Further advantages of the present invention are disposability, convenience, and comfort.

### BRIEF DESCRIPTION OF THE DRAWINGS

The above-mentioned and other features and advantages of this invention, and the manner of attaining them, will become apparent and be better understood by reference to the following description of the embodiments of the invention in conjunction with the accompanying drawings, wherein:

FIG. 1 is a plan view of the cryotherapy device of the present invention;

FIG. 2 is an exploded view of the device of FIG. 1;

FIG. 3 is a cross-sectional view of the device of FIG. 1;

FIG. 4 is a second plan view of the device of FIG. 1;

FIG. 5 is a partial cross-sectional view of the device of FIG. 1;

FIGS. 6a and 6b display examples of packaging for the device of FIG. 1; and

FIG. 7 is an isometric view of a second embodiment of the cryotherapy device of the present invention.

Corresponding reference characters indicate corresponding parts throughout the several views. The examples set out illustrate certain embodiments of the invention but do not delineate the scope of the invention.

### DETAILED DESCRIPTION

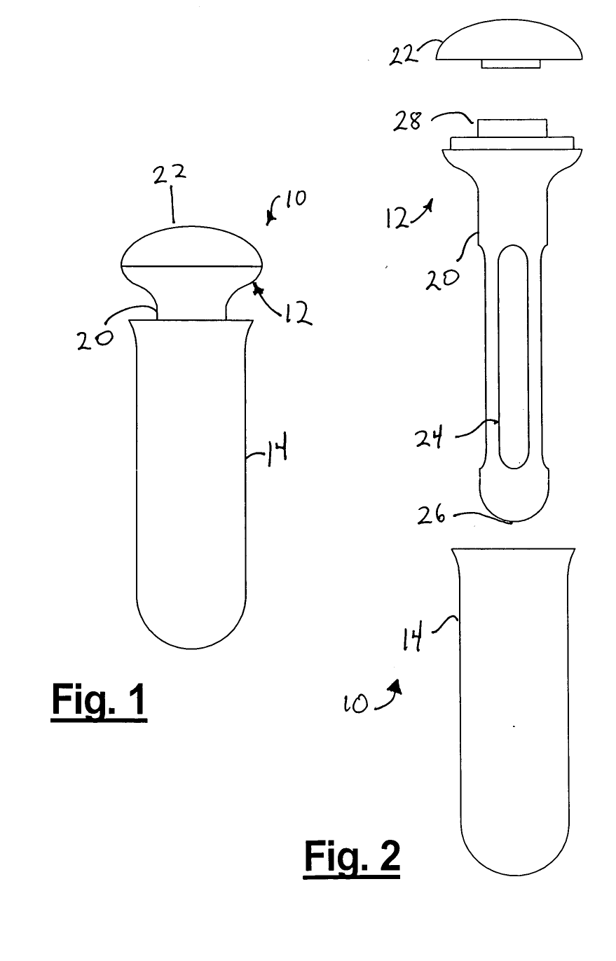

A disposable cryotherapy device for the treatment of hemorrhoids or rectal tissue after surgery of the present invention is shown in FIGS. 1 and 2. The cryotherapy device 10 includes a rectal insert 12 and a sleeve 14.

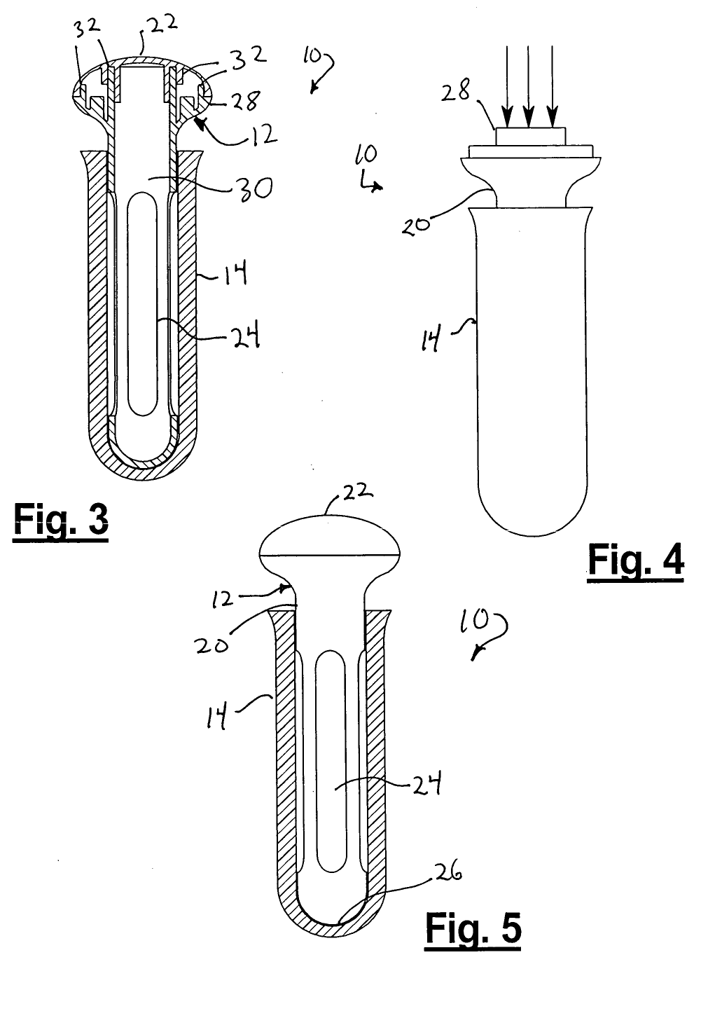

The rectal insert 12 includes a body 20 and a cap 22. The body 20 includes slots 24, rounded tip 26, and cap receiving portion 28 and encloses a chamber 30 shown in FIG. 3. The body 20 has a rounded cylindrical shape for comfortable insertion into a rectum. The cap receiving portion 28 is larger than the rest of the body 20 to prevent the rectal insert 12 from being inserted so far into the rectum that the rectal insert is difficult to remove. The chamber 30 contains a healing medium that is either frozen or at least chilled to a temperature that provides the cryotherapy effect and causes the healing medium to be thick enough that it will not flow out of the chamber 30 through slots 24. The frozen healing medium could be as simple as distilled water, or a substance such as aloe vera gel mixed with purified water, glycerin, a carbomer and a sodium hydroxide solution. Alternatively, a petroleum based mixture or an existing hemorrhoid product could be utilized as the healing medium. To maximize the cryotherapy effect, the frozen healing medium should be formulated such that it remains frozen or thickened for at least 5 minutes after the cryotherapy device 10 is applied to the rectal tissue. The healing medium preferably has a pH of about 4 to 5 in order to provide a healing medium that will retain it's thickness for the desired amount of time. The cap 22 is configured for engaging the cap receiving portion 28 of the body 20. More particularly, the cap 22 is configured to snap onto the cap receiving portion 28 of the body 20 in a sealing manner as shown in FIG. 3. The cap 22 and the cap receiving portion 28 include multiple complementary sealing structures 32 to retain the healing medium within the chamber 30. Alternatively, cap 22 and cap receiving portion 28 include complimentary threads to seal off the open end of the body 20.

The sleeve 14 fits over the rectal insert 12 such that sleeve 14 completely covers the slots 24 as shown in FIGS. 3 and 5. The sleeve 14 allows the healing medium to be placed into the chamber 30 prior to being frozen by precluding the healing medium from leaking out the slots 24.

In use, the body 20 of the rectal insert 12 is fully disposed into the sleeve 14 as shown in FIG. 4. The healing medium is placed into the chamber 30 via the cap receiving portion 28. The chamber 30 is not completely filled to allow expansion of the healing medium during changes in temperature. The cap 22 is then attached to the cap receiving portion 28.

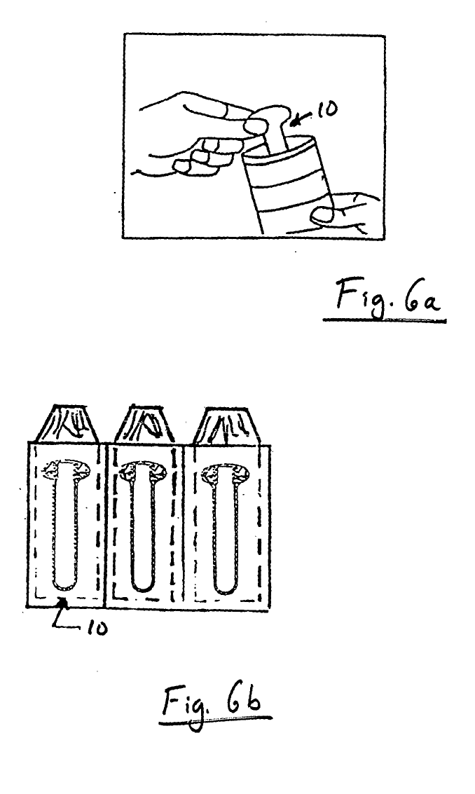

The cryotherapy device 10 is preferably individually packaged as shown in FIGS. 6a and 6b. The packages may be sealable plastic bags or a series of blow molded compartments sealed by conventional packaging methods that allow the compartments to be opened individually. Alternatively, the cryotherapy device 10 may be packaged in a bubble wrap or any other packaging method that keeps the device substantially sterile while in storage or during the freezing process.

The packaged cryotherapy device 10 containing the healing medium is placed in a freezer for a time sufficient to lower the temperature of the healing medium to the desired therapeutic temperature. The time required for freezing may vary according to the formulation and the amount of the frozen healing medium and in some instances may require lowering the temperature but not freezing the healing medium.

In order to treat hemorrhoids or rectal tissue after surgery, the packaged cryotherapy device 10 is removed from the freezer and then separated from the discardable packaging. The rectal insert 12 is removed from the sleeve 14. The healing medium does not leak out the slots 24 because it is now frozen or thickened due to chilling. The rectal insert 12 is inserted into the rectum with the rounded end 26 first. The enlarged cap receiving portion 28 and cap 22 prevent the rectal insert 12 from being inserted so far into the rectum that it is difficult to remove.

The frozen healing medium treats the tissue by cooling which can reduce inflammation and ease discomfort. Further, after several minutes, the frozen healing medium melts, thus the healing medium is delivered to the tissue providing additional treatment. The rectal insert 12 is removed from the rectum after a predetermined amount of time or until the frozen healing medium has fully melted. The rectal insert 12, the sleeve 14, and the empty package are all discarded. A string may be attached to the cap 22 to aid in the removal of the rectal insert 12 from the rectum.

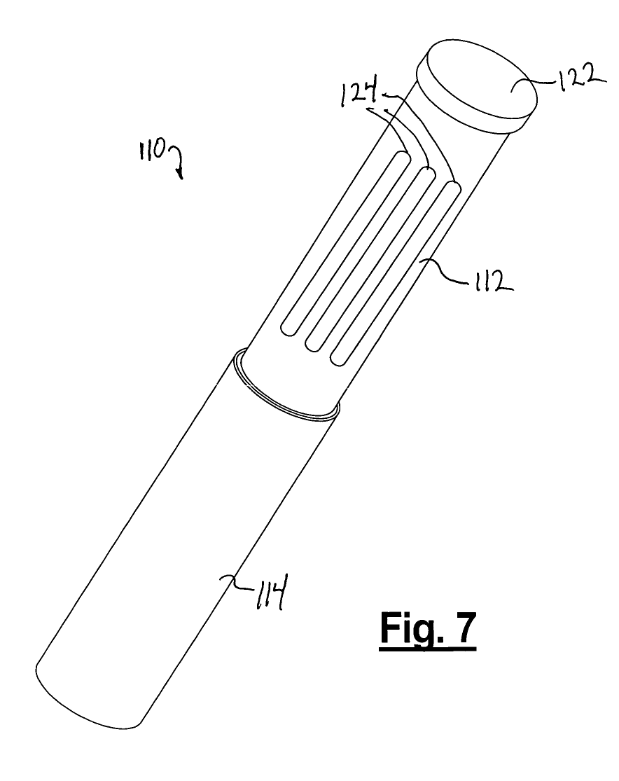

In a second embodiment, the disposable cryotherapy device is designed for topical administration. An example of a cryotherapy device 110 is shown in FIG. 7. The cryotherapy device 110 is configured to be applied externally to hemorrhoids and tissue after surgery and includes an application surface 112 having slots 124, a sleeve 114 and a cap 122. Healing medium is placed in the cryotherapy device 110 and then cooled as described in the first embodiment. The sleeve 114 is removed and the application surface 112 is placed against the external tissue to be treated and the frozen healing medium treats the tissue by cooling the tissue and subsequently melting to deliver the healing medium to the tissue. The cryotherapy device 110 is packaged individually as described in the first embodiment and is preferably disposable so the device and packaging are discarded after a single use.

It should be noted that the configuration of the cryotherapy device shown in the figures has four slots 24, however any number of slots may be used to communicate the frozen healing medium to the tissue. Further, elongated slots 24 are shown, however many acceptable shapes can be imagined.

While the invention has been described with reference to preferred embodiments, it will be understood by those skilled in the art that various changes may be made and equivalents may be substituted for elements thereof without departing from the scope of the invention. In addition, many modifications may be made to adapt a particular situation or material to the teachings of the invention without departing from the scope of the invention.

Therefore, it is intended that the invention not be limited to the particular embodiments disclosed as the best mode contemplated for carrying out this invention, but that the invention will include all embodiments falling within the scope and spirit of the appended claims.

1. A cryotherapy device for the treatment of hemorrhoids, comprising:
a hollow applicator having a wall, wherein the wall of said applicator defines a plurality of slots; and
a sleeve having an open end such that said applicator can slide into said sleeve and said sleeve covers the slots defined by the wall of said applicator.

2. The cryotherapy device of claim 1, further comprising a chilled healing medium within said hollow applicator.

3. The cryotherapy device of claim 2, wherein the healing medium comprises aloe vera gel, distilled water, glycerin, a carbomer, and a sodium hydroxide solution.

4. The cryotherapy device of claim 2, wherein the healing medium comprises an existing hemorrhoid product formulation.

5. The cryotherapy device of claim 2, wherein the healing medium is formulated to provide about 5 minutes of cooling prior to melting during treatment.

6. The cryotherapy device of claim 1, wherein said applicator includes a sealing cap.

7. The cryotherapy device of claim 6, wherein the cap and the applicator include a plurality of complementary sealing structures.

8. The cryotherapy device of claim 1, wherein said applicator is configured as a rectal insert being substantially cylindrical in shape and comprising a rounded end and an enlarged end to prevent said rectal insert from being fully inserted into a rectum.

9. The cryotherapy device of claim 1, wherein said device is disposable and individually packaged.

10. A method of treating hemorrhoids and rectal tissue, comprising:
placing an applicator having an open end and a plurality of slots into a sleeve that covers the slots, wherein the applicator contains a healing medium; and
contacting applicator to a tissue area that is to be treated.

11. The method of treating hemorrhoids of claim 10, wherein the tissue requiring treatment is rectal tissue.

12. The method of treating hemorrhoids of claim 10, further comprising sealing the open end of the applicator by affixing a cap to the open end of the applicator after said placing step.

13. The method of treating hemorrhoids of claim 12, wherein the cap and the open end of the applicator include a plurality of complementary sealing structures.

14. The method of treating hemorrhoids of claim 10, further comprising chilling the healing medium after said placing step.

15. The method of treating hemorrhoids of claim 14, wherein the healing medium is formulated to provide about 5 minutes of cooling prior to melting during said contacting step.

16. The method of treating hemorrhoids of claim 10, further comprising removing the applicator from the sleeve after said placing step.

17. The method of treating hemorrhoids of claim 10, further comprising withdrawing the applicator from the tissue area and discarding the applicator and sleeve after said contacting step.

18. The method of treating hemorrhoids of claim 10, wherein the healing medium comprises aloe vera gel, distilled water, glycerin, a carbomer, and a sodium hydroxide solution.

19. The method of treating hemorrhoids of claim 10, wherein the healing medium comprises an existing hemorrhoid product formulation.

20. A method of treating hemorrhoids and rectal tissue, comprising:
placing an applicator having an open end and a plurality of slots into a sleeve that covers the slots;
charging the applicator with a healing medium; and
contacting applicator to a tissue area that is to be treated.
